(19) United States
(12) Patent Application Publication
(10) Pub. No.: US 2004/0132712 A1
(43) Pub. Date: Jul. 8, 2004

(54) Pharmaceutical compositions comprising clavulanic acid

(76) Inventors: Otto Damon, Jenbach (AT); Herwig Jennewein, Absam (AT); Thomas Lentner, Kundl (AT); Franz Xaver Schwarz, Worgl (AT); Robert Veigl, Haag (AT)

Correspondence Address: NOVARTIS, CORPORATE INTELLECTUAL PROPERTY, ONE HEALTH PLAZA 430/2, EAST HANOVER, NJ, 07936-1080, US

(21) Appl. No.: 10/474,480

(22) PCT Filed: Apr. 11, 2002
(86) PCT No.: PCT/EP02/04062

(30) Foreign Application Priority Data
Apr. 12, 2001 (AT) 595/2001
Apr. 12, 2001 (AT) 596/2001
Apr. 12, 2001 (AT) 597/2001

(51) Int. Cl. A61K 31/397

(52) U.S. Cl. 514/210.09

(57) ABSTRACT

A pharmaceutical composition comprising an active ingredient and clavulanate wherein the clavulanate is in the form of granulated and hydrophobised particles; and intermediates in its preparation, e.g. clavulanate is granulated and/or hydrophobised form.

The present invention relates to pharmaceutical compositions comprising clavulanic acid. Clavulanic acid is a β-lactamase inhibitor, which itself is not used as a pharmaceutical, but is administered in pharmaceutical compositions together with one or more pharmaceutically active ingredients, e.g. antibiotics, in order to prevent or reduce rapid degradation of the active ingredient by bacteria at the desired site of action.

One known commercial form of a pharmaceutical composition, which contains clavulanic acid and, as an active ingredient, a β-lactam, e.g. an antibiotic, is for example AUGMENTIN, see e.g. Merck Index 12th Edition, Item 2402, e.g. containing clavulanic acid in the form of a potassium salt (K-clavulanate ) and amoxicillin in the form of a trihydrate or in the form of a salt, for example a sodium salt (see e.g. Merck Index 12th Edition, Item 617).

The processing of clavulanic acid (hereinafter also designated as “clavulanate”), e.g. in the form of a salt, e.g. a potassium salt, hereinafter designated as “K-clavulanate” in and into pharmaceutical compositions (comprising beside clavulanic acid a pharmaceutically active ingredient, e.g. a β-lactam, such as amoxicillin), is difficult, e.g. clavulanate is easily degraded and the processing behaviour of clavulanate may be unsatisfying, e.g. the bulk density of clavulanate obtained in a process for its production may be in general very low. Pharmaceutical compositions, e.g. which are intended to rapidly release the active ingredient and clavulanic acid at the site of action, include e.g. dispersible tablets and granulates for oral administration, e.g. so called “syrup granulates” and aqueous solutions, syrups, suspensions or dispersions produced therefrom. Prior to administration to patients, dispersible tablets or (syrup) granulates for oral administration are generally dissolved, dispersed or suspended in an aqueous liquid. However, clavulanic acid may rapidly degrade when brought into contact with water and, if clavulanic acid starts to dissolve too early, i.e. before reaching the intended site of action, it might be degraded. “Syrup” as used in relation with the present invention herein includes dispersions and suspensions of clavulanate in aqueous liquids, but exclude solutions of clavulanate in such aqueous liquids. Ingredients other than clavulanate in a pharmaceutical composition may be dissolved in a “syrup” derived from a “syrup granulates” as used herein.

It has now surprisingly been found that clavulanate, e.g. K-clavulanate, can be brought into a form in which clavulanate may have satisfying processing behaviours, e.g. high bulk density, and, moreover that clavulanate may be brought into a form, in which clavulanate may be protected from degradation in aqueous environment. Both findings are advantageous in a process for the production of pharmaceutical compositions comprising clavulanate.

In one aspect, the present invention provides clavulanate, e.g. K-clavulanate, in the form of granulated particles. Preferably, the granulated particles of clavulanate are auxiliary-free. “Auxiliaries” in the context of this application includes pharmaceutically acceptable exdipients such as conventionally used in pharmaceutical formulation technology, for example lubricants, e.g. magnesium stearate, mold-separating agents, e.g. talcum, binding or filling agents, e.g. polyvinyl pyrrolidone, micro-crystalline cellulose, modified starch, disintegrating agents, e.g. crosslinked carboxymethyl cellulose, crosslinked carboxymethyl starch or crosslinked polyvinyl pyrrolidone (PVPP).

Clavulanic acid (clavulanate) includes salts of clavulanic acid, e.g. alkali and earth alkali salts, preferably a potassium salt (K-clavulanate).

“In granulated form” or “in the form of granulated particles” as used herein means that clavulanate, e.g. K-clavulanate, is present in the form of granules, i.e. particles, e.g. crystalline, which are held together by van der Waals forces and by electrostatic charges. In contrast to that, in crystal agglomeration a further crystal starts to grow on a defect of another crystal and agglomerates are joined together by covalent, crystal bonds. Granulated particles generally have a higher density than agglomerates.

Granulated clavulanate, i.e. clavulanate, such as K-clavulanate, e.g. in crystalline form, consisting of particles, e.g. crystals, of clavulanic acid which are held together by van der Waals forces and by electrostatic charges, is new and may be unambigously distinguished, e.g. under a microscope such as an electron-microscope, from non-granulated clavulanate or clavulanate agglomerates. Whereas crystal-bridges representing the covalent bonds are identifiable by microscopy in agglomerated clavulanate, such bridges cannot be found in granulated clavulanate. Up to now attempts to produce clavulanate, e.g. K-clavulanate, in granulated form have not been successful.

Clavulanate in granulated form may be obtained by moist granulating of clavulanate, e.g. according to, e.g. analogously to, a moist granulation method as conventional, or, preferably, as described below:

As a starting material, K-clavulanate may be used in dry form or in solvent-moist form, preferably in solvent-moist form, e.g. comprising an amount of 0 to 5% (w/w) of solvent, e.g. in a form as obtained from its preparation process, preferably in crystalline form. K-clavulanate is most preferably obtained from n-butanol or iso-butanol with or without water as a solvent from its preparation process, e.g. such as described in WO97/18216, the content of which and the content of the literature cited therein is incorporated in the present application by reference.

In WO 97/18216, it is inter alia described that clavulanic acid may be converted into K-clavulanate in n-butanol and/or isobutanol, optionally in the presence of water, as a solvent. As the starting material for conversion, clavulanic acid may be used as such or in the form of a salt, e.g. a lithium salt or an amine salt, preferably an amine salt. Amine salts include salts of clavulanic acid as disclosed in WO 97/182216, preferably salts with tert.butylamine, tert.-octylamine (2-amino-2,4,4-trimethylpentane), N,N′-diisopropyl-ethylenediamine, N,N,N′,N′-tetramethyl-diaminoethane and 1,3-bis(dimethylamino)-2-propanol, more preferably tert.octylamine or tert.butylamine.

Clavulanic acid in the form of a salt with an amine may be produced as conventional, e.g. according to one of the methods disclosed in WO97/18216, preferably as follows:

fermenting an appropriate micro-organism, e.g. a micro-organism which is capable of producing clavulanic acid during fermentation, whereby an impure aqueous fermentation broth is obtained, which contains clavulanic acid.

optionally pre-purifying an impure aqueous fermentation broth, which contains clavulanic acid, by

removing at least part of the solids from the fermentation broth, and/or

extracting an impure or pre-purified aqueous fermentation broth, which contains clavulanic acid, with an organic solvent which is able to form two phases when in contact with water, in which organic solvent the clavulanic acid is practically insoluble or only slightly soluble under the extraction conditions; and obtaining a pre-purified aqueous fermentation broth, which contains clavulanic add;

optionally concentrating a pre-purified aqueous fermentation broth, which contains clavulanic acid,

acidifying an impure or pre-purified, optionally concentrated aqueous fermentation broth, which contains clavulanic acid, e.g. after fermentation has ended or acidifying an impure or pre-purified, aqueous fermentation broth, which contains clavulanic acid, and which has been partially harvested during fermentation, or

acidifying an aqueous solution of clavulanic acid, and obtaining an optionally concentrated, impure or pre-purified, acidified aqueous fermentation broth, which contains clavulanic acid, or obtaining an acidified, aqueous solution/suspension of clavulanic acid;

extracting an impure or pre-purified, acidified, aqueous fermentation broth, which contains clavulanic acid, or an acidified, aqueous solution/suspension of clavulanic acid, with an organic solvent, which is able to form two phases when in contact with water, in which clavulanic acid is soluble under the extraction conditions and obtaining a solution of clavulanic acid in an organic solvent;

adding an amine, preferably tert.butylamine, tert.octylamine, N,N′-diisopropylethylene-diamine, N,N,N′,N′-tetramethyl-diaminoethane or 1,3-bis(dimethylamino)-2-propanol, more preferably tert.octylamine or tert.butylamine, to a solution of clavulanic acid in an organic solvent, and obtaining clavulanic acid in the form of a salt with an amine, e.g. in the form of a solvate, e.g. an acetone solvate, and isolating clavulanic acid in the form of a salt with an amine.

The conversion of clavulanic acid in the form of an amine salt into K-clavulanate may preferably be carried out as follows:

Clavulanic acid in the form of an amine salt is dissolved in n-butanol and/or isobutanol. Water may be present in the solution, e.g. in an amount of 0.5%, preferably of 1.0% to 10%, preferably to 6%. The solution obtained, optionally treated with activated carbon, is brought Into contact with a source of potassium ions which is capable of forming K-clavulanate with clavulanic acid. Cation sources of this kind are described e.g. in WO97/18216 (in therein cited literature) and include preferably potassium salts of a (C₂₋₈)-carboxylic acid, e.g. 2-ethylhexane-carboxylic acid, for example potassium acetate. If a potassium acetate is used, acetic add may additionally be added during the reaction. Contact of the source of potassium ions with the solution of an amine salt of clavulanic acid may take place according to one of the methods disclosed in WO97/18216 (in therein cited literature) and is preferably effected as follows:

A solution of the source of potassium ions in a solvent, preferably in n-butanol and/or isobutanol, e.g. containing water, is added, e.g. in portions, to the solution of the amine salt of clavulanic acid. At least one equivalent of the source of potassium ions, preferably 1.0 to 3.0, e.g. about 1.1 to about 2.0 equivalents, may be used per mol of clavulanic acid (salt). K-clavulanate may precipitate from the reaction mixture. For example, in order to complete precipitation, a further solvent, in which K-clavulanate is poorly soluble (anti-solvent) may be added to the mixture, and/or the mixture obtained may be cooled, e.g. to temperatures of a range of about −5° C. to about 10° C., such as about 0° C. to about 5° C. K-clavulanate is isolated, e.g. by filtration or centrifugation, and is obtained in solid, solvent-moist form, e.g. in crystalline form, e.g. comprising between 0 and 5% (w/w) of solvent.

In moist granulation of dry or solvent-moist K-clavulanate, a granulation liquid may be used to obtain a granulation mass. A granulation liquid includes water or an organic solvent, or an organic solvent mixed with water, preferably water or an organic solvent mixed with water. In a granulation liquid an organic solvent is preferably an alcohol, including e.g. ethanol, n-butanol, Isobutanol, preferably a mixture comprising n-butanol or isobutanol and containing 0.5 to 10% (v/v), e.g. 1.0 to 6% (v/v) of water.

A granulation mass suitable for moist granulation may be obtained by mixing a granulation liquid with K-clavulanate. The amount of granulating liquid is not critical and the minimum amount of granulating liquid may be easily determined. A granulation mass preferably contains K-clavulanate and granulating liquid in an amount of 5% (w/w based on wet mass), preferably of 6% (w/w) to 25% (w/w), preferably to 20% (w/w). In one embodiment the obtained granulation mass is dried and granulated K-clavulanate is obtained. In another embodiment the granulation mass is extruded to obtain granulated K-clavulanate. Preferably, the granulation mass is extruded, e.g. according, e.g. analogously, to conventional extruding methods, e.g. at appropriate extrusion temperatures, e.g. including temperatures from room temperature and below, e.g. 0° C. to 10° C. The obtained extruded mass is dried and granulated K-clavulanate is obtained, or the extruded mass is passed through a sieve, preferably the extruded mass s passed through a sieve. A preferred mesh size of the sieve is in the range of 1.0 mm to 4.0 mm, e.g. in the range of 2.0 mm to 3.0 mm.

A sieved extruded mass obtained by such a method is dried to obtain granulated K-clavulanate. Alternatively a (sieved) extruded mass may be (further) diminuished, e.g. according to, e.g. analogously, to a method as conventional, e.g. using a fast-action blade.

The granulation mass or the extruded mass, which is optionally sieved and/or further diminuished, undergoes a drying process. High temperatures may degrade clavulanic acid and suitable drying conditions may be found by preliminary tests. Preferably a rapid pre-drying of the granulation mass or (sieved) extruded mass and gentle after-drying is carried out. Pre-drying may be effected by passing a gas, e.g. air, through the mass at temperatures in th range of room temperature and above, e.g. at temperatures of 25° C. to 50° C., preferably 25° C. to 40° C. Pre-drying preferably continues until the drying substrate has temperatures at or below room temperature, e.g. 25° C. or less, for example 10° C. to 25° C., preferably 15° C. to 25° C. Drying may be carried out according to, e.g. analogously to, a method as conventional, e.g. by convection drying such as vacuum drying or dry-air drying. Suitable drying operations are effected as conventional such as by fluidized bed drying or by conveyor-belt-drying, e.g. in a shelf-dryer, a tray-dryer or a chamber-dryer. Pre-drying is preferably effected by belt drying or fluidised bed drying, more preferably fluidised bed drying. For after-drying, dry-air drying is preferably used.

Granulated K-clavulanate is obtained upon drying. Granulated K-clavulanate optionally may be broken up to obtain granulated K-clavulanate (particles) with a desired particle size, e.g. having a desired distribution of grain size, e.g. according, e.g. analogously, to a method as conventional, e.g. by use of a sieve, mill or a compacting device. A desired distribution of grain size may depend on a desired further processing. Preferably, no auxiliary is added during the whole process of moist-granulating K-clavulanate in order to obtain auxiliary-free, granulated K-clavulanate particles.

In another aspect the present invention provides a process for the production of K-clavulanate in the form of granulated particles comprising subjecting K-clavulanate to a moist granulation method.

In a further aspect, the present invention provides a process for the production of K-clavulanate in the form of granulated particles comprising the steps

a. moistening K-clavulanate with a granulating liquid to obtain a granulation mass,

b. optionally extruding the granulation mass obtained to form an extruded mass,

c. optionally passing, e.g. pressing, the extruded mass through a sieve,

d. drying the granulation mass or (sieved) extruded mass, and

e. diminuishing the size grain of of the granulate obtained.

Preferably, no auxiliary is added during the process to obtain K-clavulanate in the form of granules.

In another aspect the present invention provides K-clavulanate in the form of granules produced by a process comprising subjecting K-clavulanate to a moist granulation method, e.g. by a process comprising steps a. to e. as described herein above.

Granulated K-clavulanate may have advantageous processing properties, e.g.

high bulk densities, e.g. 0.5 to 0.8 g/ml, such as 0.6 to 0.7 g/ml, for example 0.61 to 0.7 g/ml

high abrasive resistance.

High bulk densities indicate in general advantageous high flowability properties. High abrasive resistance indicates in general high stability. Both properties may be strived for in processing procedures. Bulk density and abrasive resistance are e.g. determined by methods indicated in pharmacopoeiias.

In another aspect the present invention provides K-clavulanate particles, e.g. consisting of granulated K-clavulanate particles, having a bulk density of 0.5 g/ml to 0.8 g/ml.

K-clavulanate obtained by a moist granulation as described above may have excellent advantageous processing properties, which may simplify the processing of K-clavulanate into pharmaceutical compositions containing K-clavulanate. For example, K-clavulanate may be provided in high bulk density and may thus be readily compressed. Consequently small tablets may be obtained comprising a high proportion of K-clavulanate. It was also surprisingly found that granulated K-clavulanate may be obtained in high purity, e.g. practically in the same purity as K-clavulanate used in moist granulation even though K-clavulanate during processing may easily undergo degradation reactions.

In another aspect the present invention provides the use of clavulanate, e.g. K-clavulanate, in the form of granulated particles in the production of a pharmaceutical composition. A further aspect of the present invention is a pharmaceutical composition comprising clavulanate e.g. K-clavulanate, together with an active ingredient, e.g. beside pharmaceutically acceptable excipient(s), which composition is characterised in that the clavulanate is present in the form of granulated, preferably auxiliary-free particles.

In another aspect the present invention provides clavulanate, e.g. K-clavulanate, e.g. in the form of granulated particles, in the form of hydrophobised particles, e.g. said particles comprising an oil and a hydrophobic solid, e.g. a composition comprising, e.g. consisting, of clavulanate, an oil and a hydrophobic solid.

Hydrophobised particles of clavulanate are designated hereinafter also as “hydrophobised clavulanate”. “Hydrophobising” means that the clavulanate particles are protected from rapid dissolution in aqueous liquids at pH values that are different from those at the site of activity, with the result that degradation of clavulanate may be reduced or prevented in aqueous compositions. According to the present invention, it was found that hydrophobised clavulanate particles practically do not start to dissolve in aqueous liquids, e.g. such as liquids used in oral administration, if clavulanate particles have been treated with an oil and with a hydrophobic solid, e.g. if clavulate particles are coated with an oil and with a hydrophobic solid.

An oil includes pharmaceutically acceptable oils, for example paraffin oils and silicone oils, preferably silicone oils, e.g. silicone oils which have antifoaming characteristics, e.g. siloxanes, such as dimethylpolysiloxane. The oil may be present as such or in a mixture with further auxiliaries. Appropriate auxiliaries include e.g. flow-improving agents, e.g. silicon dioxides, e.g. highly dispersed SiO₂, such as Aerosil®. Hydrophobic solids include e.g. magnesium stearate. The ratio of amounts of clavulanic acid:oil:hydrophobic solid is not critical. The minimum amount of oil and hydrophobic solid, which prevent dissolving, may be easily determined by preliminary tests. Conveniently 0.05 g to 0.3 g of oil and 0.05 g to 0.3 g of hydrophobic solid per gram of K-clavulanate may be used.

Hydrophobised clavulanate may be produced by mixing clavulanate with an oil and a hydrophobic solid.

Clavulanate, e.g. K-clavulanate, may be used in a hydrophobisation process in a particle form in which it is obtained by a production process, preferably clavulanate in granulated form is used. Mixing may be effected in conventional mixers, e.g. by use of forced-flow mixers. Preferably, clavulanate is pre-mixed with the oil, and the resulting mixture is mixed with the hydrophobic solid. A homogeneous mixture may be and should be obtained.

Hydrophobised clavulanate, namely particles comprising clavulanate, e.g. granulated K-clavulanate, together with an oil and a hydrophobic solid, e.g. particles coated with a (homogeneous) mixture of the oil and the hydrophobic solid, are obtained. Clavulanate particles should not stick together under the mixing conditions and appropriate non-sticking-conditions may be easily determined, e.g. by preliminary testing. We have found that hydrophobised clavulanate may be stable in aqueous liquids, e.g water, aqueous suspensions, dispersions, salvia, i.e. clavulanate in hydrophobised clavulanate is practically not degraded in aqueous environment. However, hydrophobised clavulanate is still well absorbed within the gastro-intestinal-tract in order to deliberate clavulanate at its site of activity, i.e. the bacterial beta-lactamases.

In another aspect the present invention provides a process for the production of hydrophobised clavulanate, e.g. K-clavulanate, e.g. in the form of granulated particles, comprising mixing clavulanate with an oil and a hydrophobic solid. Surprisingly it was also found that the advantageous processing properties of preferably auxiliary-free, granulated K-clavulanate according to the present invention may be maintained after hydrophobisation, e.g. if hydrophobisation is carried out under conditions in which the granulated, hydrophobised K-clavulanate particles do not stick together, granulated, hydrophobised K-clavulanate particles may maintain high abrasive resistance and high bulk density, e.g. e.g. 0.5 to 0.8 g/ml, such as 0.6 to 0.7 g/ml. The hydrophobised clavulanate particles obtained from such mixing process may be used as such in pharmaceutical compositions.

In another aspect the present invention provides the use of clavulanate, e.g. K-clavulanate, such as clavulanate in granulated form, in the form of hydrophobised particles for the production of pharmaceutical compositions, and, in another aspect a pharmaceutical composition comprising clavulanate e.g. K-clavulanate, together with an active ingredient, e.g. beside pharmaceutically acceptable excipient(s), which composition is characterised in that the clavulanate is present in the form of hydrophobised particles.

In another aspect, the present invention provides a pharmaceutical composition comprising clavulanate, e.g. K-clavulanate, together with a pharmaceutically active ingredient, e.g. beside pharmaceutically acceptable excipient(s), which composition is characterised in that the clavulanate is present in the form of granulated and hydrophobised particles.

A pharmaceutically active ingredient in a pharmaceutical composition according to the present invention is preferably one or more, more preferably one, antibiotic, e.g. a β-lactam, such as amoxicillin. In a pharmaceutical composition according to the present invention amoxicillin may be present in free form, for example in the form of a solvate, e.g. a trihydrate, or in the form of a salt, e.g. a sodium salt, and is preferably present in the form of a trihydrate.

Pharmaceutical compositions according to the present invention preferably contain pharmaceutically acceptable excipient(s) (auxiliary(ies)), including for example carrier(s), diluent(s), and may be produced as appropriate, e.g. according, e.g. analogously, to a method as conventional, but using granulated and/or hydrophobised particles of clavulanate.

A pharmaceutical composition according to the present invention is in solid form and is an oral pharmaceutical composition, e.g. a pharmaceutical composition for oral administration, and is preferably a tablet, e.g. a dispersible tablet or a (syrup) granulate for oral adminstration, e.g. for the production of aqueous suspensions or dispersions.

A pharmaceutical composition according to the present invention may be obtained as appropriate, e.g. according, such as analogously, to a method as conventional und is preferably obtained as follows:

Hydrophobised K-clavulanate, which is in granulated or non-granulated form, is mixed with amoxicillin and optionally with pharmaceutically acceptable excipients. The mixture obtained may be used as such, e.g. in the form of (syrup) granulates, or the mixture obtained is further processed, e.g. compressed to obtain tablets, e.g. dispersible tablets.

A pharmaceutically active ingredient, such as a β-lactam, e.g. amoxicillin, is preferably used in the form of auxiliary-free agglomerates, e.g. as described in WO97/33564. The content of WO97/33564 is incorporated into the present application by reference.

In WO97/33564 auxiliary-free agglomerates of a β-lactam, inter alia amoxicillin in the form of a trihydrate, are described, having for example an average volume-based grain size of 100 μm to 1000 μm, preferably 400 μm to 600 μm, such as 200 m to 600 μm; having for example the following grain size distribution:

| | | |
|---|---|---|
| <100 μm: | 1% to 30%, | e.g. 5% to 20% |
| 100 μm to 500 μm: | 10% to 80%, | e.g. 20% to 60% |
| 500 μm to 1000 μm: | 10% to 80%, | e.g. 25% to 60% |
| >1000 μm: | max. 30%, | e.g. max. 15% |
| >2000 μm: | max. 0.5%, | e.g. max. 0.1%; |

and/or having a bulk density of 0.4 g/ml to 0.8 g/ml, for example 0.5 g/ml to 0.7 g/ml. Such agglomerates may be obtained e.g. by

forming into a paste a solid β-lactam antibiotic e.g. in the form of a powder, with an average volume-based grain size of 10 μm to 30 μm, with about the following distribution of grain size:

| | |
|---|---|
| 4 μm to 80 μm | 80% |
| >125 μm | 1 to 5% |

and a bulk density of 0.15 g/ml to 0.45 g/ml, as usually obtained in the production process for a β-lactam, with a liquid in which the β-lactam antibiotic is insoluble or slightly soluble,

kneading and extruding the paste in a double-screwed extruder having a specific mechanical energy input of 0.01 kilowatt-hour/kg to 0.1 kilowatt-hour/kg at temperatures in the range of 10° C. to 80° C., and

drying agglomerates obtained.

Appropriate liquids include e.g. water, alcohols and mixtures thereof; as well as organic solvents such as acetone. An alcohol may preferably be ethanol or iso-propanol. The amount of liquid may be appropriate to result in a kneadable paste with the β-lactam antibiotic and may be preferably as follows (expressed in % by weight, based on the paste): 3 to 20, preferably 5 to 10 for the case that the active ingredient is slightly dissolved by the liquid; and 5 to 35, preferably 10 to 20 for the case that the active ingredient is insoluble in the liquid. The β-lactam may be placed into the extruder in an already moist form, or in dry form. If the βlactam is placed into the extruder in dry form the liquid may be dispensed into the extruder simultaneously with the β-lactam antibiotic. The preferred specific mechanical energy input used for processing includes 0.02 kilowatt-hour/kg to 0.6 kilowatt-hour/kg. The optimum degree of density of the β-lactam antibiotic agglomerates may be such that mechanical stability of the agglomerates is appropriate, i.e., after drying, the agglomerates should not disintegrate into a powder because this would negatively affect the free-flow capability. But the agglomerates should not be extremely mechanical stable (density too high), because extreme stable agglomerates would not be prone to form mechanically stable tablets during the tablet formation process. The optimum degree of density corresponds exactly to the observed maximum torque pick-up on the extrusion screw which passes through during extrusion as the amount of liquid increases. Thus, the optimum degree of density of the powder is easily controllable. In Example 3 of WO97/33564 it is described:

Acetone-moist amoxicillin trihydrate (10% to 15% acetone based on moist mass) is agglomerated in a double-screwed extruder (process length 3 D) at 150 kg/h at a maximum torque pick-up of the extrusion screws of 25% to 35%. The screws are configured with conveyer elements and right-and left-handed kneading blocks. After drying the extruded moist mass in a fluidized bed drier, agglomerates of amoxicillin trihydrate (yield 99.9%) having the following properties are obtained:

Distribution of grain size

| | |
|---|---|
| <100 μm: | 13% |
| 100 μm to 500 μm: | 71% |
| 500 μm to 1000 μm: | 12% |
| >1000 μm: | 4% |

Bulk density: 0.56 g/ml; stamped density: 0.67 gml.

In a further aspect, the present invention provides a pharmaceutical composition, e.g. a dispersible tablet or a granulate for oral adminstration, comprising clavulanate, e.g. K-clavulanate, together with a pharmaceutically active β-lactam, e.g. amoxicillin, e.g. in the form of a trihydrate, e.g. beside pharmaceutically acceptable excipient(s), which composition is characterised in that the clavulanate is present in the form of granulated and hydrophobised particles and which is further characterized in that the β-lactam is in the form of auxiliary-free agglomerates.

Pharmaceutically acceptable excipient(s) include one or more excipient(s), e.g. excipient(s) as conventional in formulation technology in the production of pharmaceutical compositions, e.g. including colorants, sweeteners, glidants and lubricants, fillers, flavours or disintegrants. Such pharmaceutical compositions may be produced by appropriate methods, e.g. according, e.g. analogously, to a method as conventional but using optionally granulated, hydrophobised K-clavulanate and using a β-lactam in the form of auxiliary-free agglomerates.

Preferably such pharmaceutical compositions are produced by mixing optionally granulated, preferably auxiliary-free, hydrophobised K-clavulanate with amoxicillin trihydrate in the form of auxiliary-free agglomerates and with excipient(s) to obtain a granulate for oral administration, and optionally compressing the resulting mixture, in order to obtain tablets, e.g. dispersible tablets. A preferred pharmaceutical composition is a dispersible tablet.

In a further aspect, the present invention provides a process for the production of dispersible tablets comprising K-clavulanate together with amoxicillin in the form of a trihydrate beside pharmaceutically acceptable excipient(s), which process comprises mixing K-clavulanate in the form of granulated and hydrophobised particles with amoxicillin in the form of a trihydrate in the form of auxiliary-free agglomerates and pharmaceutically acceptable excipient(s) and compressing the mixture obtained to produce dispersible tablets.

The weight ratio of amoxicillin and clavulanic acid in a pharmaceutical composition of the present invention may be from 2:1 to 30:1, e.g. 2:1, 4:1, 5:1, 7:1, 8:1, 14:1, 16:1, 20:1. In a dispersible tablet or in a granulate for oral administration said weight ratio is preferably 7:1 or 8:1. A dispersible tablet is preferably a so-called “1 gram tablet”, which contains as an active ingredient amoxicillin in the form of a trihydrate in an amount which corresponds to an amount of 875 mg±40 mg amoxicillin, e.g. in the form of auxiliary-free agglomerates, and which contains granulated and hydrophobised K-clavulanate in an amount corresponding to 125 mg±6 mg, respectively, clavulanic acid; together with pharmaceutically acceptable auxiliaries.

In another aspect the present invention provides a dispersible tablet or a granulate for oral administration, comprising K-clavulanate in the form of granulated and hydrophobised particles and amoxicillin in the form of a trihydrate in the form of auxiliary-free agglomerates wherein the weight ratio of amoxicillin (unsolvated amoxicillin) and clavulanic acid is 7:1 or 8:1, amoxicillin beside pharmaceutically acceptable excipient(s).

In the following examples, all temperatures are in degree Centigrade and are uncorrected. The following abbreviations are used:

K-clavulanate: clavulanic acid in the form of a potassium salt

In the TABLES, the following abbreviations are used:

EX: example number

T_{M}: casing temperature of the mixer in ° C.

SOLV: granulation liquid, which is used for granulation to obtain the granulation mass

% SOLV: amount of granulation liquid in % w/w of the total granulation mass. The percentages in parenthesis in the column % SOLV indicate the water portion in % v/v in the granulation liquid

DRY: type of drying used

DEGR: loss of K-clavulanate content, which is determined (HPLC) in % after granulation and drying, based on the K-clavulanate content before granulation and drying

COL: coloration of K-clavulanate after granulation and drying, compared with the K-clavulanate before granulation and drying. “NO” in the column COL indicates none, “YES” indicates colouration

T_{z}: air inlet temperature in the fluidised bed drier in ° C.

T_{G}: temperature of the dried substrate in the fluidised bed drier in ° C.

SD: bulk density in g/ml

N.D.: not detected by method used (HPLC)

### I. EXAMPLES FOR THE PRODUCTION OF K-CLAVULANATE IN GRANULATED FORM

### Process 1

K-clavulanate is mixed with the granulating liquid in a mixer with a cooled casing and the granulation mass obtained is dried.

Drying A)a): Dry air is passed through a container having a perforated bottom.

Drying A)b): Pre-drying in a fluidised bed drier at 30° or 40° air inlet temperature. When the dry substrate has reached a temperature of below 25° C., after-drying is effected by passing dry air through the container with a perforated bottom.

Upon drying K-clavulanate is obtained in granulated form. The granulate obtained is broken over a sieve of mesh size 1 mm.

Results are as set out in TABLE 1 below:

TABLE 1

| EX | T_{M} | SOLV | % SOLV | DRY | DEGR | COL |
|---|---|---|---|---|---|---|
| 1 | 4 | H₂O | 7 | A)a) | 2.4 | YES |
| 2 | 4 | EtOH (50%) | 11 | A)b) | 1.3 | NO |
| 3 | 3 | n-Butanol (4%) | 20 | A)b) | 1.3 | NO |
| 4 | 3 | n-Butanol (4%) | 20 | A)b) | 0.6 | NO |

The granulate obtained according to examples 1 to 4 has higher bulk density than the K-clavulanate used as starting material.

### Process 2

K-clavulanate is mixed with n-butanol containing 4% water in a mixer having a cooled casing (3° C.) and a granulation mass is obtained. The granulation mass is extruded through an extruder (screw extruder).

The extruded mass obtained is dried. Pre-drying is effected in a fluidised bed drier at 30° or 40° air inlet temperature T_{Z} until reaching a temperature T_{G} of the dried substrate, and after-drying is carried out by passing through dry air. K-clavulanate is obtained in granulated form. No colouration of granulated K-clavulanate occurs compared with K-clavulanate before granulation and drying. The granulate obtained is broken over a sieve of mesh size 1.0 mm.

Results are as set out in TABLE 2 below:

TABLE 2

| EX | % SOLV | T_{Z} | T_{G} | DEGR | SD |
|---|---|---|---|---|---|
| 5 | 16 | 30 | 21 | N.D. | 0.57 |
| 6 | 17 | 40 | 26 | N.D. | 0.44 |
| 7 | 19 | 30 | 22 | 0.8 | 0.64 |

K-clavulanate in granulated form obtained according to examples 5 to 7 has a higher bulk density than K-clavulanate used as starting material.

### Process 3

K-clavulanate is mixed with n-butanol containing 4% water in a mixer having a cooled casing (2°), a granulation mass is obtained and extruded through an extruder (screw extruder). The extruded mass obtained is pressed through a sieve of mesh size 2 mm or 2.5 mm and the sieved extruded mass obtained is dried. Pre-drying is effected in a fluidised bed drier at 30° until reaching a temperature of the dried substrate of 22°, and after-drying is carried out by passing through dry air.

K-clavulanate is obtained in granulated form. No colouration of granulated K-clavulanate occurs compared with K-clavulanate before granulation and drying.

The granulate obtained is broken over a sieve having a mesh size of

a 0.8 mm

b 1.0 mm, or

c 1.5 mm.

K-clavulanate is obtained in granulated form with a bulk density in the case of

a. is of 0.63 g/ml

b. is of 0.64 g/ml, and

c. is of 0.67 g/ml.

K-clavulanate in granulated form obtained has a higher bulk density compared with K-clavulanate before granulation. The K-clavulanate content in K-clavulanate in granulated form obtained is reduced by 0.1% compared with the K-clavulanate content before granulation.

### II. EXAMPLE FOR THE PRODUCTION OF K-CLAVULANATE IN GRANULATE AND HYDROPHOBISED FORM

148.6 g of K-clavulanate in granulated form, obtained according to a method of examples 1, are mixed with 17.9 g of dimethylpolysiloxane and 17.9 g of magnesium stearate in a forced-flow mixer. Particles of K-clavulanate in granulated and hydrophobised form are obtained. Depending on the bulk density of K-clavulanate in granulated form used as a starting material the hydrophobised particles obtained have a granulate-corresponding bulk density of 0.5 to 0.8 g/ml.

The hydrophobised clavulanic acid particles are suspended in aqueous liquids (water). Degradation of clavulanic acid in the hydrophobised particles of that suspension was determined and it was found that practically no degradation of clavulanic acid occurred in that aqueous environment.

### III. EXAMPLE FOR THE PRODUCTION OF A DISPERSIBLE TABLET COMPRISING K-CLAVULANATE AND AMOXICILLIN IN THE FORM OF A TRIHYDRATE

The amounts refer to the amounts per tablet: 148.6 mg of K-clavulanate in granulated form is hydrophobised with 17.9 mg of dimethylpolysiloxane and 17.9 mg of magnesium stearate according to the method described in Example II. The granulated and hydrophobised K-clavulanate particles obtained are mixed at 15 rpm in a free-fall mixer with 1011.1 mg of amoxicillin in the form of a trihydrate in auxiliary-free, agglomerated form, 1.8 mg of iron oxide, 7.1 mg of aspartame, 12.5 mg of magnesium stearate, 28.6 mg of aromatic substances, 28.6 mg of highly dispersed silicon dioxide and 171.6 mg of a crosslinked homopolymer of a polyvinyl pyrrolidone (Polyplasdone XL 499®). A mixture is obtained, which is pressed into stable tablets.

When such a tablet is added to water the tablet disperses and a suspension is produced within a short time. Hydrophobised K-clavulanate particles are released from the tablet. Degradation of clavulanic acid in the hydrophobised particles of that suspension was determined and it was found that practically no degradation of clavulanic acid occurred in that aqueous environment

1. A pharmaceutical composition comprising clavulanate together with a pharmaceutically active ingredient, which composition is characterised in that the clavulanate is present in the form of granulated and hydrophobised particles.

2. A pharmaceutical composition according to claim 1 comprising clavulanate together with a pharmaceutically active β-lactam which composition is characterised in that the clavulanate is present in the form of granulated and hydrophobised particles and that the β-lactam is in the form of auxiliary-free agglomerates.

3. A pharmaceutical composition according to any one of claims 1 or 2 which is a dispersible tablet or a granulate for oral administration, comprising K-clavulanate in the form of granulated and hydrophobised particles and amoxicillin in the form of a trihydrate in the form of auxiliary-free agglomerates wherein the weight ratio of amoxicillin (unsolvated amoxicillin) and clavulanic acid is 7:1 or 8:1, beside pharmaceutically acceptable excipient(s).

4. Clavulanate in the form of hydrophobised particles.

5. Clavulanate according to claim 4, wherein the hydrophobised particles are granulated.

6. Use of clavulanate according to any one of claims 5 or 6 for the production of pharmaceutical compositions.

7. A pharmaceutical composition comprising clavulanate according to any one of claims 4 or 5 together with an active ingredient beside pharmaceutically acceptable exdpient(s).

8. Clavulanate in the form of granulated, auxiliary-free particles.

9. Granulated, auxiliary-free-K-clavulanate particles having a bulk density of 0.5 g/ml to 0.8 g/ml.

10. Use of clavulanate according to any one of claims 8 or 9 in the production of a pharmaceutical composition.

11. A pharmaceutical composition comprising clavulanate together with an active ingredient beside pharmaceutically acceptable excipient(s), which composition is characterised in that the clavulanate is present in the form of granulated, auxiliary-free particles.
